(19) United States
(12) Patent Application Publication
(10) Pub. No.: US 2013/0189771 A1
(43) Pub. Date: Jul. 25, 2013

(54) CELL CULTURE DEVICE

(71) Applicants: Hans Strandberg, Sundbyberg (SE); Lars Enochson, Molndal (SE); Joakim Sandstedt, Goteborg (SE)

(72) Inventors: Hans Strandberg, Sundbyberg (SE); Lars Enochson, Molndal (SE); Joakim Sandstedt, Goteborg (SE)

(21) Appl. No.: 13/358,014

(22) Filed: Jan. 25, 2012

(51) Int. Cl. C12M 1/00

(52) U.S. Cl. 435/289.1

(57) ABSTRACT

A cell culture device has a chamber bottom, a wall section and a lid that enclose a culture chamber. A first electrode in the chamber bottom extends over a cell culture area of the culture chamber onto which cells are to be cultured. A second electrode in the lid is aligned with the first electrode and extends over an area in the lid corresponding to the cell culture area. A stimulation pulse generator applies stimulation pulses over the electrodes to form an electric field with a homogenous current density in the cell culture area.

### BACKGROUND OF THE INVENTION

1. Field of the Invention

The present invention generally relate to a cell culture device, and in particular to such a cell culture device that can be used to expose cultured cells to electric stimulation.

2. Description of the Prior Art

Heart diseases, such as heart failure and ischemia, are very common medical conditions and are among the most common causes of death in the industrial world.

Many of the heart diseases can be treated or combated by revascularization, pharmacological treatment and/or implanting a pacemaker or intracardiac cardioverter-defibrillator (ICD) into a patient to thereby provide pacing and/or shocking therapy to the patient's heart. However, if the heart disease has caused injury to the cardomyocytes, a damaged region will still be present in the heart since the cardiomyocytes are generally unable to regenerate to a sufficient degree in an adult heart. Thus, for some patients the heart might be so damaged that the above-mentioned treatment approaches are not sufficient. In clear contrast, a cardiac transplantation is required. There is, though, a shortage of donor hearts and this put limits to the number of patients that can be treated through cardiac transplantation.

An alternative approach would be to replace the damaged myocardial tissue to thereby restore cardiac function. Much research has been put into the field of tissue engineering to, in vitro, generate cardiomyocytes that could be transplanted into the patient to there replace the killed cardiomyocytes. Different approaches have been suggested in the art to generate cardiomyocytes from human stem cells, including human embryonic stem cells and human mesenchymal stem cells. The stem cells are, at least partly during the in vitro culturing, exposed to electrical field stimulation to trigger cardiac differentiation towards cardiomyocytes. In the art, the main focus has been to select the particular culture conditions, including culture medium, additives, selection of support cells, etc. Although much effort and research have been put into finding suitable culture conditions, far less focus has been invested in the design and function of the cell culture devices or bioreactors that are used for the cell culturing and differentiation. In clear contrast, often traditional bioreactors have been reused and equipped with electrodes to apply the electric field stimulation. However, the electric fields and the current density profiles obtained in such prior art bioreactors are far from optimal for cell differentiation point of view.

Serena, et al., Electrical stimulation of human embryonic stem cells: Cardiac differentiation and the generation of reactive oxygen species, Experimental Cell Research (2009), 315: 3611-3619 uses a custom design bioreactor formed as an array of 4×4 wells in polydimethylsiloxane (PDMS). Circular electrodes of 1.3 mm diameter are inserted at two sides of each row of wells.

US 2007/0238169 A1 discloses a bioreactor for cell culturing and differentiation. Electrodes in the form of conductive gold wires are provided along opposite sides of a cell growing plate with cells grown therebetween.

There is a need for a cell culture device that is designed to effectively promote differentiation of cultured cells towards cardiomyocytes or cells having characteristics of spontaneous contraction similar to cardiomyocytes.

### SUMMARY OF THE INVENTION

It is a general objective to provide to cell culture device capable of providing even current density in a cell culture area.

This and other objectives are met by embodiments disclosed herein.

An aspect of the embodiments relates to a cell culture device comprising a chamber bottom with an attached wall section delimiting a culture chamber having a cell culture area onto which cells are to be cultured. A lid is attachable to the wall section to enclose the culture chamber. A first electrode is provided in or attached to the chamber bottom to extend over at least the cell culture chamber. A corresponding second electrode is provided in or attached to the lid and is aligned with the first electrode when the lid is attached to the wall section. The second electrode extends over an area in the lid that substantially corresponds to at least the cell culture area. The first electrode and the second electrode are connectable to a stimulation pulse generator configured to apply stimulation pulses over the electrodes to form an electric field between the electrodes. The formed electric field has a current density level within a target current density interval in the cell culture area.

The particular arrangement of the electrodes together with the design of the cell culture device enable formation of a very even current density in the cell culture area where the cells will be present. The even current density implies that the cells will be exposed to a sufficient but still acceptable current density to trigger contraction or differentiation towards contracting ability. Thus, areas in the culture chamber either having too low current density that are not sufficient to trigger contraction or induce differentiation or have too high current density that is harmful for the cells are minimized.

### BRIEF DESCRIPTION OF THE DRAWINGS

FIG. 1 is a view from above of a cell culture device according to prior art.

FIG. 2 is a cross-sectional view of the cell culture device illustrated in FIG. 1.

FIG. 3 illustrates a current density profile achieved in a cell culture device illustrated in FIG. 1.

FIG. 4 is a cross-sectional view of a cell culture device according to an embodiment.

FIG. 5 illustrates a current density profile achieved in a cell culture device illustrated in FIG. 4.

FIG. 6 is an illustration of an embodiment of an electrode in the cell culture device.

FIG. 7 is an illustration of another embodiment of an electrode in the cell culture device.

FIG. 8 is an illustration of a further embodiment of an electrode in the cell culture device.

FIG. 9 is a cross-sectional view of a cell culture device according to prior art.

FIG. 10 illustrates a current density profile achieved in a cell culture device illustrated in FIG. 9.

FIG. 11 is a cross-sectional view of a cell culture device according to another embodiment.

FIG. 12 is a cross-sectional view of a cell culture device according to a further embodiment.

FIG. 13 is a cross-sectional view of a cell culture device according to yet another embodiment.

FIG. 14 is a cross-sectional view of a cell culture device according to a further embodiment.

### DESCRIPTION OF THE PREFERRED EMBODIMENTS

The embodiments generally relate to a cell culture device or bioreactor that is designed to apply stimulation pulses to cells present in the cell culture device. The cell culture device has been designed to present optimal current density profile and distribution to achieve optimal stimulation of the cultured cells.

The embodiments are based on the finding that prior art cell culture devices having stimulation functionality are not designed with regard to the current density profile to which the cultured cells are exposed. In clear contrast, very little effort has been put into the prior art of analyzing the current density profile achieved in the cell culture devices and the effect the current density profile has on the cultured cells.

Thus, analysis of prior art cell culture devices surprisingly showed, as is presented herein, that the current density profiles of the prior art cell culture devices cause extensive cell death in some part of the cell culture area and too low current amplitudes in other part of the cell culture area. Hence, the effective part of the cell culture area in which cells are exposed to acceptable and sufficient current density levels merely constitute a portion and sometimes only a minor portion of the total cell culture area. This means that the efficiency of prior art cell culture devices is surprisingly low with the regard to the percentage of the cultured cells that are viable and have been exposed to desired current density levels after application of stimulation pulses.

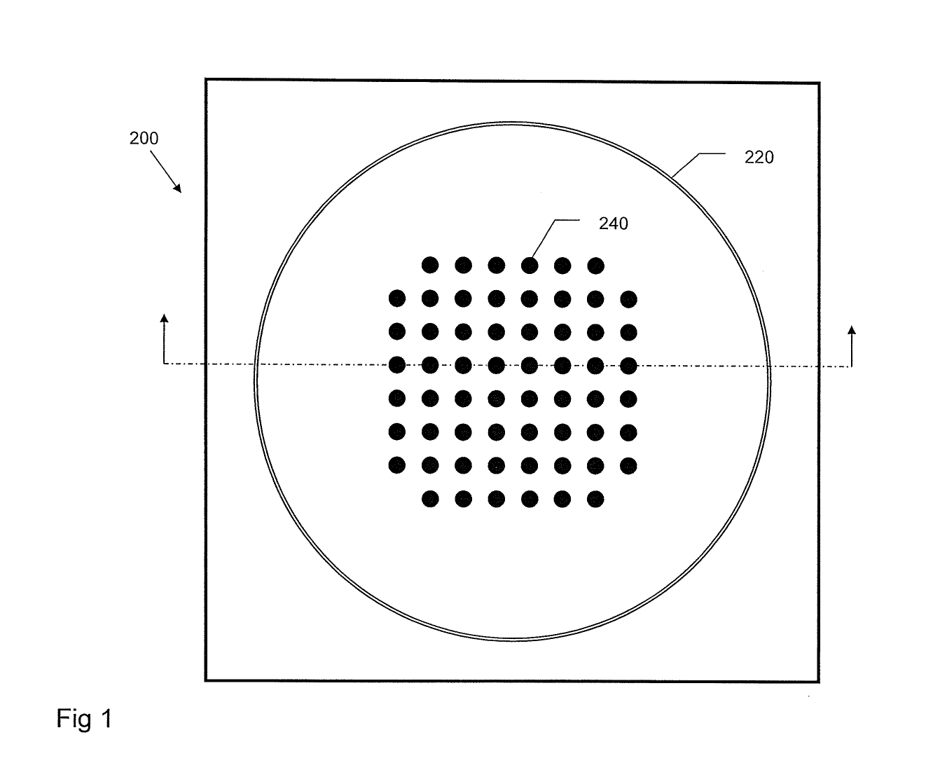

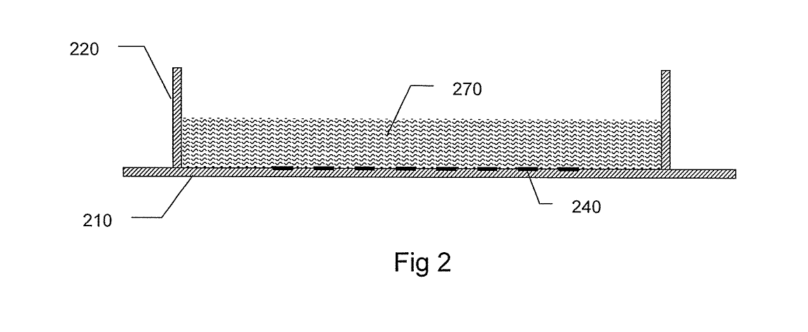

FIG. 1 is a view from above of a cell culture device 200 currently available on the market and marketed as a planar Multielectrode Array (MEA) by Multi Channel Systems, Reutlingen, Germany. The cell culture device 200 basically consists of, which is more clearly shown in the cross-sectional view in FIG. 2, a chamber bottom 210 housing an array or grid of individual electrodes 240. Each such electrode 240 has a respective conductive path to external connectors (not shown) to electrically connect the electrodes 240 to a pulse generator (not shown) to apply stimulation pulses to the electrodes 240. The chamber bottom 210 is connected to a wall section 220 in the form of an open cylinder to thereby delimit a culture chamber comprising cells placed on the surface of the chamber bottom 210 and a culture medium 270 that is conductive. The wall section 220 and the chamber bottom 210 are made of glass and the electrodes 240 are made of titanium nitride and have a diameter in the range of 10-100 μm and an electrode-to-electrode distance in order of 30-500 μm.

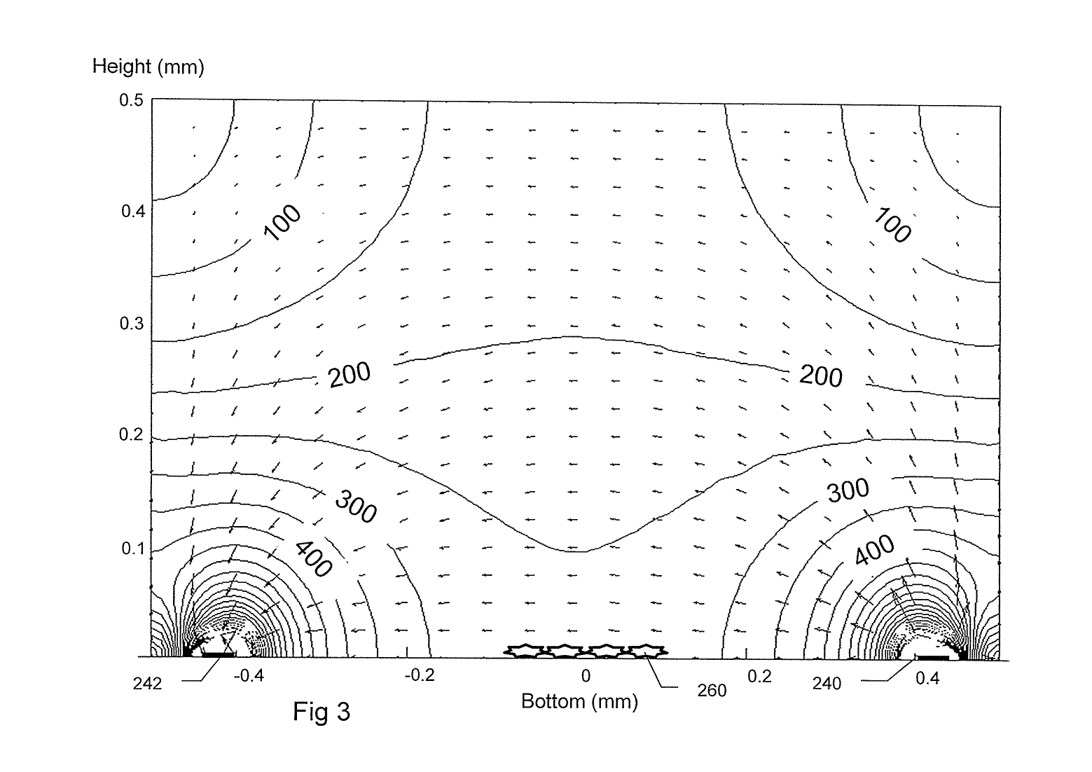

FIG. 3 illustrates a current density profile achieved with the prior art cell culture device 200 illustrated in FIGS. 1 and 2. Only two of the electrodes 240, 242 are shown in FIG. 3 as black boxes at the bottom of the drawing. The applied voltages are 0 V and 1 V. The culture medium 270 in the culture chamber has a conductivity of 0.4 S/m. The current between the electrodes 240, 242 flow as indicated by the small arrows and the current density is shown by the curve lines and values (100-400) given in A/m². Suitable current density levels for stimulating cells 260 cultured on the top surface of the chamber bottom 210 are typically in the interval 150-400 A/m². Below 150 A/m² the current density is too low in order to stimulate the cultured cells 260, whereas a current density above 400 A/m² will harm and even kill the cells 260.

It is easily seen from FIG. 3 that the current density distribution is highly uneven with this prior art cell culture device 200. Close to the electrodes 240, 242 the current density is deadly high for the cells 240. Further away from the electrodes 240, 242 the current density drops rapidly so the main area will not reach enough current density level for stimulation. Thus, there is typically only a small region around each electrode 240, 242 having appropriate current density level for inducing stimulation in the cultured cells 260.

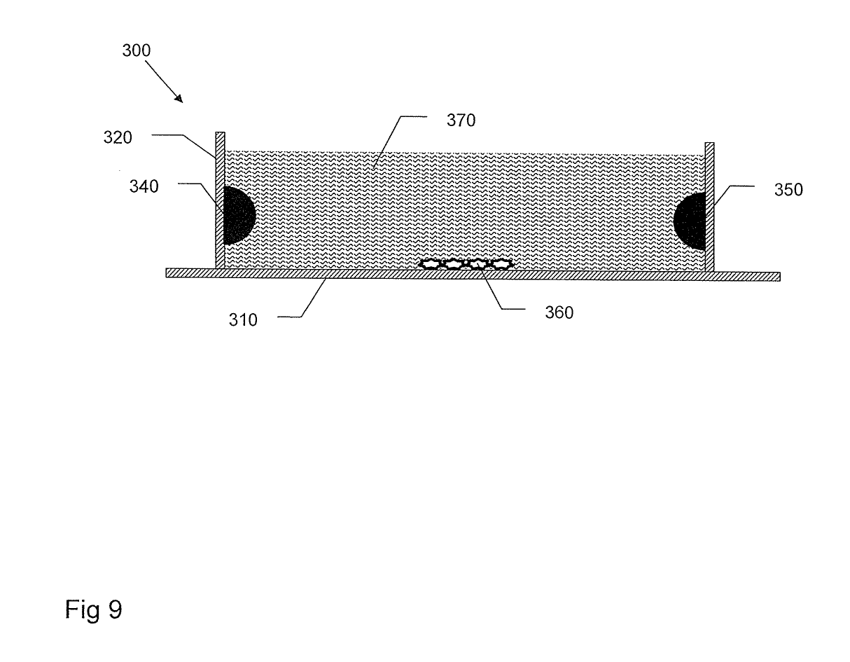

FIG. 9 is a cross-sectional view of another cell culture device 300 according to prior art. This cell culture device 300 comprises a chamber bottom 310 connected to a wall section 320 that encloses a culture chamber comprising the culture medium 370. Cells 360 are attached to the top surface of the chamber bottom 310. This cell culture device 300 basically corresponds to the one used in Serena, et al., Electrical stimulation of human embryonic stem cells: Cardiac differentiation and the generation of reactive oxygen species, Experimental Cell Research (2009), 315: 3611-3619. A culture chamber has a size of about 5 mm with the electrodes 340, 350 molded into two opposite wall sections 320. Each electrode 340, 350 is in the form of a rod of diameter 1.3 mm running along the side of the culture chamber.

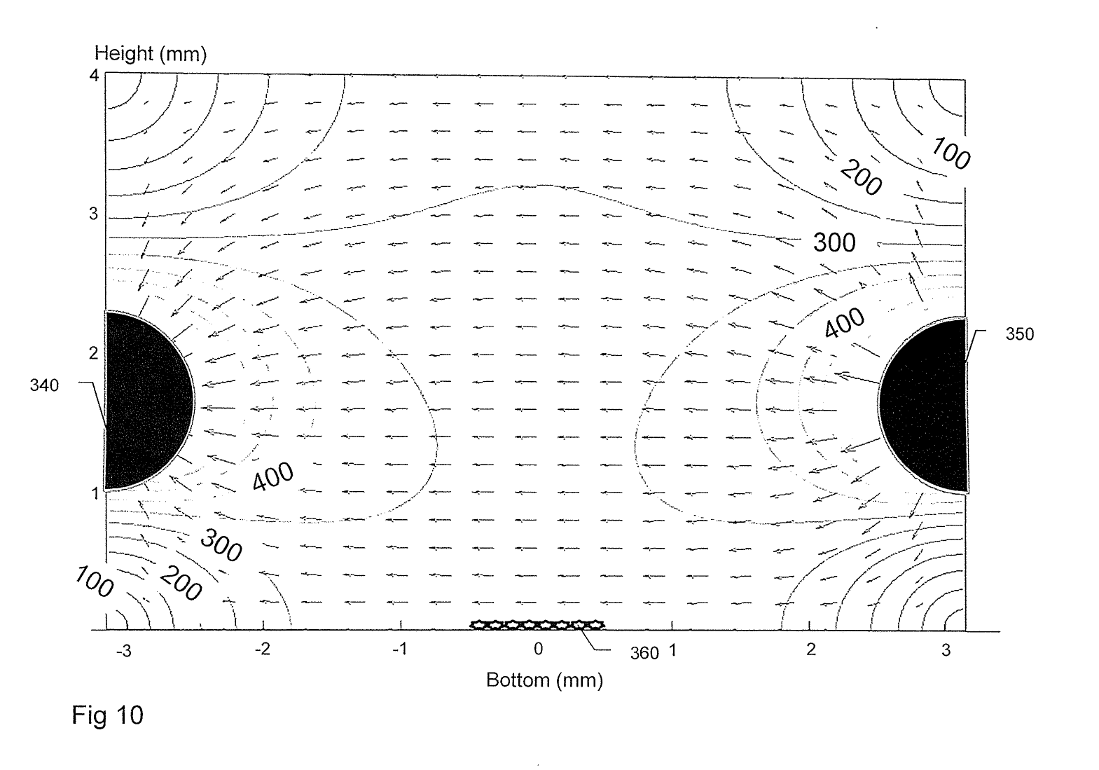

FIG. 10 illustrates the current density profile achieved in the cell culture device 300 of FIG. 9 with an applied voltage of 0 V and 5 V and a culture medium having a conductivity of 0.4 S/m. The current between the electrodes 340, 350 will flow as indicated by the arrows and the current density is shown by the curves and values (100-400) given in A/m².

The current density distribution in FIG. 10 is somewhat more even as compared to the one in FIG. 3. However, there is still only a limited area of the chamber bottom 310 that can be used for stimulation of cells 360 with sufficient but not too high current density.

Thus, the prior art cell culture devices 200, 300, as represented by FIGS. 1, 2 and 9, are not designed from current density point of view. In fact, the distribution of current density within a culture chamber has not been identified as an important issue within the technical field. Hence, no efforts have been made to design cell culture devices that maximize the cell culture area in the culture chamber where cells can be cultured and stimulated with sufficiently high but still acceptable current densities.

A further, related problem with the prior art cell culture devices 300 and in particular the one illustrated in FIG. 9 is that relative high voltages are required in order to reach sufficient current densities in at least a portion of the cell culture area in which the cells 360 are present. Voltages in the order of one or two volts will not cause any serious problems with regard to electrochemical properties at the electrodes 340, 350 for many metallic materials. However, if the voltage of the applied stimulation pulses increases to five volt as in the above identified article in Experimental Cell Research, corrosion and/or oxidation will occur at the electrodes 340, 350. Such high voltages restrict the length of the time period during which stimulation pulses can be applied by the cell culture device 300 and the type of metallic material that can be used for designing the electrodes 340, 350.

The cell culture device of the embodiments has been designed to not have these unexpected shortcomings and problems of the prior art. Thus, the cell culture device is able to produce an even and homogenous current density within a large cell culture area to thereby be able to correctly stimulate the vast majority of cells cultured in the cell culture area. Hence, the efficiency of the cell culture device in terms of the quantity of the cultured cells that are exposed to sufficiently high but acceptable current densities will be higher as compared to the prior art.

The cell culture device of the embodiments can be used for any type of in vitro culturing of cells where there is a desire to apply stimulating pulses to the cultured cells. For instance, the cells could be cardiomyocytes or other types of cells that are capable of contracting in response to applied stimulation pulses. The cell culture device could then be used in in vitro experiments with such cardiomyocytes or other contracting cells, for instance with the purpose of identifying suitable stimulation pulse profiles or stimulation conditions. The results of the in vitro experiments could then used to design implantable medical devices (IMDs), such as pacemakers or implantable cardioverter-defibrillators (ICDs), and in particular the pacing or stimulation pulses generated by such IMDs.

The cell culture device could, thus, be used for culturing cells that are already capable of contracting in response to applied stimulation pulses, for instance HL-1 cells, which is a cardiac muscle cell line that contracts and retains phenotypic characteristics of the adult cardiomyocyte (Claycomb, et al., HL-1 cells: A cardiac muscle cell line that contracts and retains phenotypic characteristics of the adult cardiomyocyte, Proceedings of the National Academy of Sciences of the United States of America (1998), 95: 2979-2984).

The cell culture device can also be used to induce contractility in cells by exposing the cells to stimulation pulses. For instance stem cells, including embryonic stem cells and mesenchymal stem cells, can be differentiated in vitro into cardiomyocytes or at least into cells having the capability of contracting in response to applied stimulation pulses. Thus, by culturing these cells under conditions and in a cell culture device where the cells are exposed to stimulation pulses the cells will induce cardiac differentiation and cause inducible contractibility.

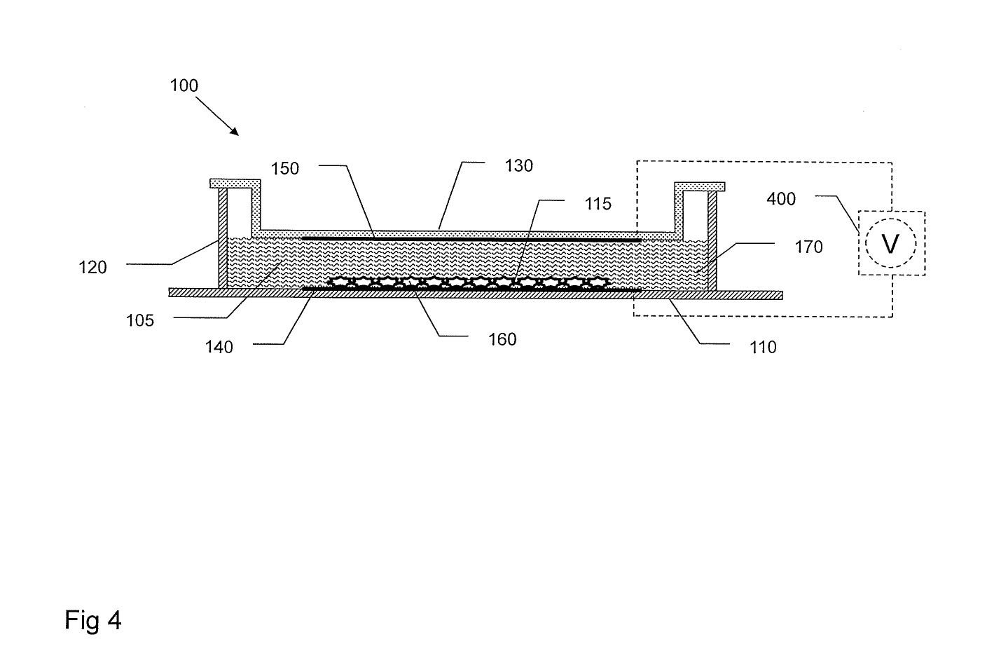

FIG. 4 is a cross-sectional view of an embodiment of a cell culture device 100. The cell culture device 100 comprises a chamber bottom 110. A wall section 120 is attached to the chamber bottom 110 and delimits a culture chamber 105. The wall section 120 could be in the form of a single wall, such as in the form of an open cylinder having a first end attached to the chamber bottom 110. In an alternative embodiment, the wall section 120 could be in the form of multiple separate but inter-connected walls delimiting, for instance, a triangular, quadratic or rectangular, culture chamber 105. The number of including walls in the wall section 120 and how these are interconnected are not relevant to the design of the cell culture device 100 as long as the wall section 120 delimits a culture chamber 105 designed to contain culture medium 170 and cells 160.

The cell culture device 100 also comprises a lid 130 that is attachable to the wall section 120 to enclose the culture chamber 105. Thus, when the lid 130 is attached to the wall section 120 the culture chamber 105 is enclosed by the lid 130, the wall section 120 and the chamber bottom 110. The lid 130 is preferably removably attachable to the wall section 120. Thus, the lid 130 can be attached onto the wall section 120 to enclose the culture chamber 105 and later on be removed from the wall section 120 to get access to the culture chamber 105 and the cells 160 cultured therein.

In an embodiment, the wall section 120 has a first end connected and attached to the chamber bottom 110. In such a case, the lid 130 could be attached to a second, opposite end of the wall section 120 as indicated in FIG. 4. Alternatively, the lid 130 could be attached to the inner side(s) or outer side(s) of the wall section 120.

The lid 130 could be simply placed onto the wall section 120 without the need for any special locking solution. Alternatively, various locking solutions, such as snap-fit locks, could be used to secure the lid 130 onto the wall section 120.

The cell culture device 100 and its culture chamber 105 comprises at least one cell culture area 115 onto which cells 160 are to be cultured. In the embodiment illustrated in FIG. 4, the cell culture area 115 is present on the surface of the chamber bottom 110.

The cell culture device 100 also comprises a first or bottom electrode 140 provided in or attached to the chamber bottom 110. This first electrode 140 extends over at least the cell culture area 115 in the chamber bottom 110. This means that the first electrode 140 could have an outer perimeter that is aligned with the outer perimeter of the cell culture area 115. In this embodiment, the first electrode 140 has basically the same extension as the cell culture area 115. In another embodiment, the first electrode 140 extends beyond the perimeter of the cell culture area 115.

The first electrode 140 preferably has the same general shape as the shape of the culture chamber 105, i.e. preferably circular, elliptical, triangular, quadratic or rectangular, depending on the particular design of the wall section 120.

The first electrode 140 could be provided in the chamber bottom 110. In such a case, the first electrode 140 could be enclosed inside the chamber bottom 110 and thereby be surrounded by the material of the chamber bottom 110. In an alternative embodiment, the first electrode 140 is attached to one of the main (planar) sides of the chamber bottom 110. For instance, the first electrode 140 could be arranged on the top surface of the chamber bottom 110, i.e. the surface facing the culture chamber 105. The chamber bottom 110 may have an indentation in its top surface. In such a case, the first electrode 140 could be arranged in that indentation. The top surface of the first electrode 140 is then preferably aligned with the top surface of the bottom chamber 110 to form one continuous surface facing the culture chamber 105. In an alternative approach, the first electrode 140 is attached to the bottom surface of the chamber bottom 110, i.e. the surface opposite to the above-mentioned top surface. The first electrode 140 may then be attached to this bottom surface or be placed in an indentation present in the bottom surface.

The cell culture device 100 also comprises a second, or top or lid electrode 150 provided in or attached to the lid 130. The second electrode 150 is arranged in the lid 130 to be aligned with the first electrode 140 when the lid 130 is attached to the wall section 120 and the culture chamber 105 is enclosed by the lid 130, the wall section 120 and the chamber bottom 110. Thus, the second electrode 150 thereby becomes positioned above the first electrode 140 and the two electrodes 140, 150 are aligned.

The second electrode 150 extends over an area in the lid 130 that substantially corresponds to at least the cell culture area 115 in the culture chamber 105. Thus, second electrode 150 has an extension along its main surface that at least matches the extension of the cell culture area 115. Hence, when the lid 130 is attached to the wall section 120 the second electrode 150 will cover the cell culture area 115.

As mentioned above, the second electrode 150 extends over an area in the lid 130 substantially corresponding to at least the cell culture area 115. “Substantially corresponding” means that the extension of the second electrode 150 is preferably at least equally large as the extension of the cell culture area 115. The second electrode 150 could, though, have an extension that is somewhat smaller than the extension of the cell culture area 115 and still the cell culture device 100 will be able to present a homogenous current density in the cell culture area 115. Thus, “substantially corresponding” also encompasses the case where the extension of the second electrode 150 is preferably equal to or larger than 95% of the extension of the cell culture area 115, preferably at least 97% and more preferably at least 98%, such as at least 99% of the extension of the cell culture area 115.

The second electrode 150 is provided in or attached to the lid 130. This means that the second electrode 150 could be enclosed inside the lid 130 or attached to one of the main (top or bottom) surfaces of the lid 130, optionally present in an indentation in a main surface, in correspondence to the first electrode 140 and the bottom surface 110.

The first electrode 140 and the second electrode 150 are connectable to a stimulation pulse generator 400 that is configured to apply stimulation pulses over the first electrode 140 and the second electrode 150. By applying stimulation pulses between the electrodes 140, 150 an electric field is formed between the first electrode 140 and the second electrode 150. The particular design of the first electrode 140 and the second electrode 150 relative to the cell culture area 115 implies that the formed electric field will have a current density level within a target current density interval in the cell culture area 115. Thus, the current density level achieved in the cell culture area 115 where the cells 160 are present will be limited to be within the target current density interval. This means that the current density distribution in the culture chamber 105 will be even and homogenous at least in the cell culture area 115.

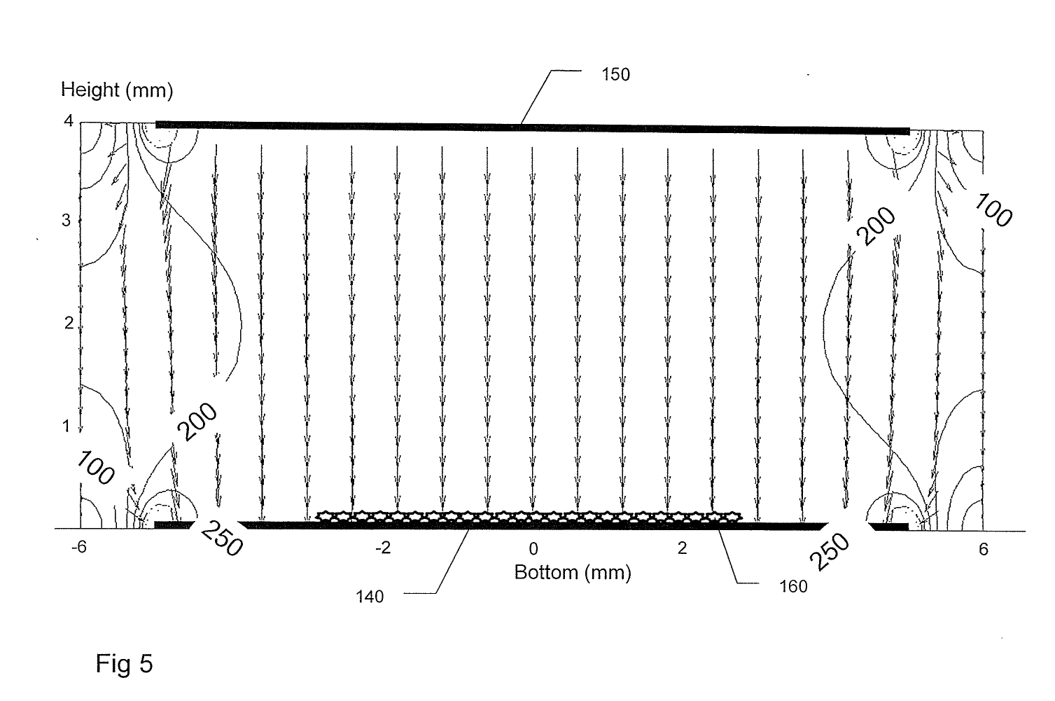

FIG. 5 illustrates the current density profile achieved with the cell culture device 100 as illustrated in FIG. 4 when voltages of 0 V and 2 V are applied to the electrodes 140, 150 and when the culture chamber 105 is filled with a culture medium 170 having a conductivity of 0.4 S/m. The current flow between the electrodes 140, 150 is indicated with arrows and the current density is shown by curves and current density values (100-250) in A/m². The current density is evenly distributed in the cell culture area 115 in which the cells 160 are present. Only at the edges of the electrodes 140, 150 is the current density non-optimal for stimulation of the cells 160. Hence, the vast majority of the area of the enclosed culture chamber 105 can be used for cell culturing and stimulation with optimal current densities, i.e. within the target current density interval.

The first electrode 140 provided in or attached to the chamber bottom 110 is preferably in the form of a plate electrode of an electrically conductive material. The first electrode 140 is therefore advantageously in the form of a thin plate or sheet, i.e. having a significantly larger extension, e.g. diameter or sides, as compared to its thickness. The thickness of the first electrode 140 can in fact be so thin that the first electrode 140 is basically in the form of a foil of the electrically conductive material.

In a particular embodiment, the plate electrode is attached to the chamber bottom 110 so that the surface of the first electrode 140 facing the culture chamber 105 constitutes a cell culture surface of the cell culture area 115. Hence, in this embodiment the cells are cultured on the first electrode 140.

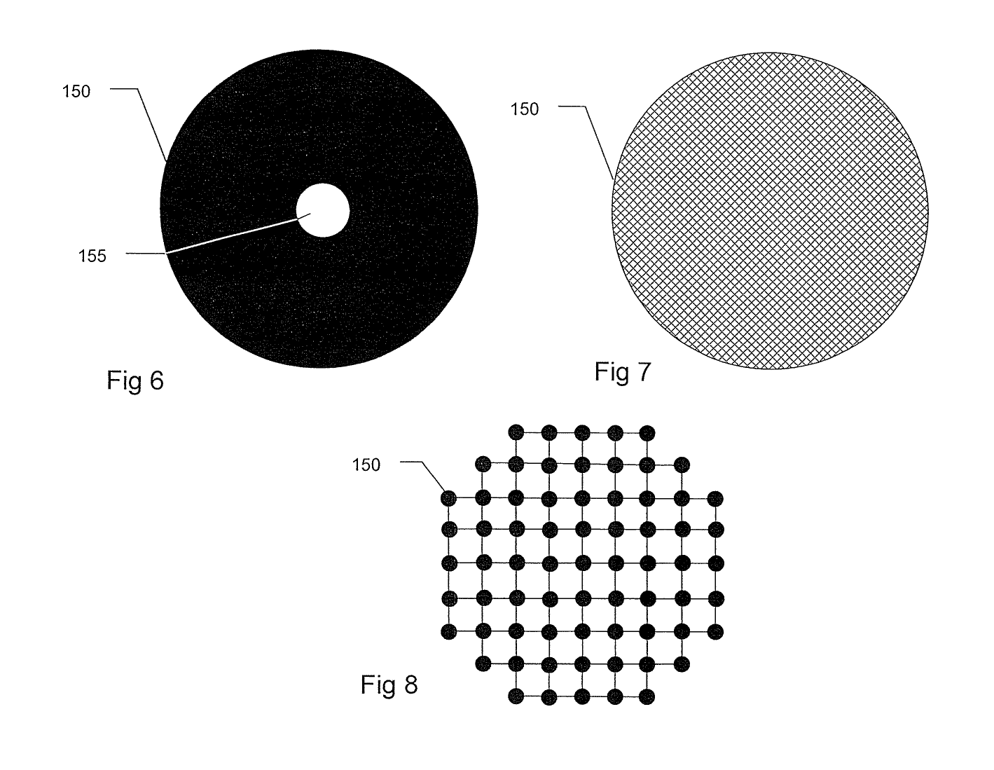

FIGS. 6 to 8 illustrate different embodiments of the second electrode 150 that can be used in the cell culture device 100. In all these embodiments, a circular geometry has been assumed for the second electrode 150 to be used in a cell culture device 100 with a wall section 120 in the form of an open cylinder. If another geometry than a circle is used for the wall section 120, the shape of the second electrode 150 preferably matches this another geometry. A circular second electrode 150 should therefore only be seen as an illustrative but non-limiting example of second electrode 150 according to the embodiments.

In an embodiment as disclosed in FIG. 6, the second electrode 150 is a plate electrode of an electrically conductive material provided in or attached to the lid 130. The second electrode 150 is then in the form of a thin plate or sheet in similarity to the first electrode 140 discussed in the foregoing. The plate or sheet could be very thin so that the second electrode 150 basically is in the form of a foil of the electrically conductive material.

If the first and second electrodes 140, 150 are in the form of two plate electrodes, visual inspection into the culture chamber 105 is basically restricted to through the wall section 120 (if transparent) or at most to the outer peripheral part of the chamber bottom 110 and the lid 130 (if transparent) unless the first electrode 140 and the second electrode 150 extend over substantially the whole area of the culture chamber 105.

It is, though, generally preferred to be able to visually inspect the cells 160 when present in cell culture area 115 of the culture chamber 105 by microscopy. This is possible if at least one of the first electrode 140 and the second electrode 150 comprises at least one through hole 155. In such a case, visual inspect, including microscopy, is possible into the culture chamber 105. In a particular embodiment both the first electrode 140 and the second electrode 150 comprises at least one respective hole 155. The at least one through hole 155 in the second electrode 150 is then arranged in the second electrode 150 to be aligned with the at least one through hole in the first electrode 140 when the lid 130 is attached to the wall section 120. Visual inspection is then possible through the through holes 155 even by microscopy. The first and second electrode 140, 150 could comprise a single through hole 155 each or multiple, i.e. at least two, through holes 155 each.

The presence of the through holes 155 in the electrodes 140, 150 should not significantly negatively affect the current density distribution in the cell culture area 115. Hence, the through holes 155 are preferably rather small as compared to the size of the electrodes 140, 150. In a particular embodiment, the longest dimension of a through hole 155, i.e. diameter of a circular through hole or side length of a quadratic/rectangular through hole, is preferably smaller than the distance between the first electrode 140 and the second electrode 150 when the lid 130 is attached to the wall section 120. If the through hole dimension is limited by this distance between the two electrodes 140, 150 the presence of the through holes 155 will not significantly negatively affect the current density distribution in the cell culture area 115.

In a particular embodiment, the first and second electrodes 140, 150 are plate electrodes but lacking the through hole 155 of FIG. 6.

FIG. 7 illustrates another embodiment of the second electrode 150 that enables visual access through the second electrode 150. The second electrode 150 is here in the form of a net of an electrically conductive material provided in or attached to the lid 130. Visual access through the second electrode 150 is possible through the meshes in the net. A further embodiment is illustrated in FIG. 8 where the second electrode 150 consists of an array, matrix or grid of electrically inter-connected dots of an electrically conductive material provided in or attached to the lid 130. The dots are then preferably arranged sufficiently densely in the lid 130 to form an electric field between the first electrode 140 and the second electrode 150 that have a current density level within the target current density interval in the cell culture area 115.

The designs of the second electrode 150 shown in FIGS. 6-8 can also be used for the first electrode 140, which then could be in the form of a plate electrode, a net or an array, matrix or grid of electrically inter-connected dots of the electrically conductive material.

The electrically conductive material used for the first electrode 140 and the second electrode 150 could be same type of material or different materials can be used in the two electrodes 140, 150. It is though generally preferred to use the same electrically conductive material in the two electrodes 140, 150 and in particular using a metallic material that is electrically conductive. Non-limiting, but preferred, examples of such electrically conductive material include rhodium, palladium, iridium, platinum, tantalum, gold, titanium, titanium nitride and alloys of rhodium, palladium, iridium, platinum, tantalum, gold and titanium.

The chamber bottom 110, the wall section 120 and the lid 130 of the cell culture device 100 are made of an electrically insulating material. The electrically insulating material of the chamber 110 and the lid 130 and preferably also of the wall section 120 is preferably transparent. Examples of such electrically insulating materials that can be used are glass and plastic material and in particular transparent glass and plastic materials. Any of the electrically insulating materials commonly used within the technical field for designing cell culture devices can be used according to the embodiments.

With reference anew to FIG. 4, the first electrode 140 could be designed to only extend over the cell culture area 115 in the culture chamber 105 as shown in FIG. 4. In an alternative embodiment, the first electrode 140 extends substantially over the whole culture chamber 105 or indeed over the whole chamber bottom 110. The chamber bottom 110 could basically correspond to the bottom of the culture chamber 105, i.e. the chamber bottom 110 does not extend beyond the wall section 120. Alternatively, the chamber bottom 110 could extend beyond the wall section 120 as shown in FIG. 4. It is, though, sufficient that the first electrode 140 extends substantially over the whole cell culture area 115 but advantageously extends over the whole bottom of the culture chamber 105. In the latter case, the complete bottom of the culture chamber 105 could be used as cell culture area 115, thereby maximizing the number of cells 160 that can be cultured and stimulated in the cell culture device 100.

The second electrode 150 preferably extends over an area in the lid 130 that corresponds to the area that the first electrode 140 extends over in the chamber bottom 110. Thus, the first and second electrodes 140, 150 are preferably equally large and extend over the same part of the culture chamber 105 when aligned.

As is well known in the art, the culture medium 170 in the cell culture device 100 is preferably exchanged if the culturing of the cells 160 extends over time. In an embodiment, the exchange of the culture medium 170 can be performed simply by removing the lid 130 from the wall section 120 to get access to the culture chamber 105 and the culture medium 170. The culture medium 170 or at least a part thereof can then be pipetted or sucked away and replaced by fresh culture medium 170. The lid 130 is then once more attached to the wall section 120 to enclose the culture chamber 105.

The lid 130 could alternatively be equipped with openings through which a pipette or other instrument can be inserted into the culture medium 170 in order to remove old culture medium 170 and replace it with fresh culture medium 170. Any such opening is then preferably provided in a peripheral part of the lid 130 away from the cell culture area 115 to minimize the risk of unintentionally contacting the cultured cells 160 with the pipette or instrument.

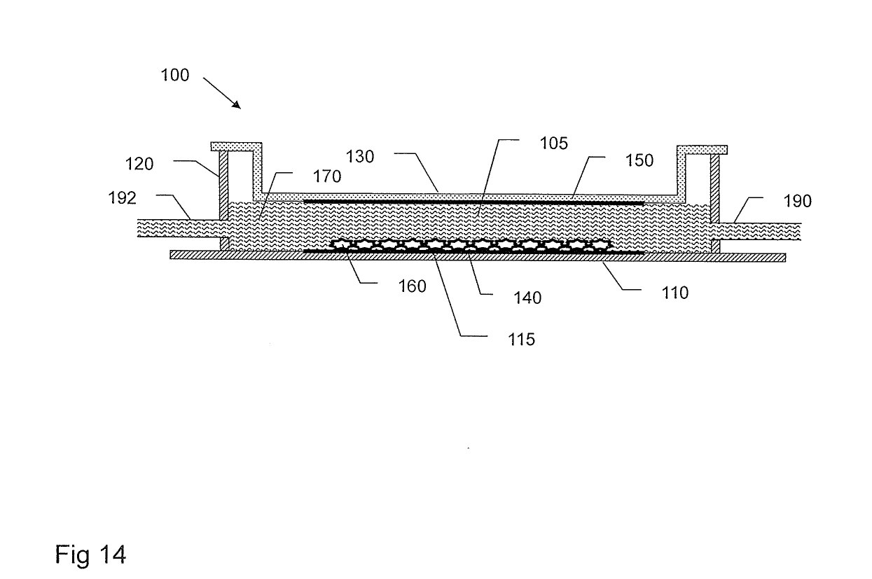

FIG. 14 is an illustration of an embodiment of the cell culture device 100 that enables continuous exchange of culture medium 170 without the need of removing the lid 130. The cell culture device 100 then comprises a flow inlet 190 arranged in the wall section 120 to input culture medium 170 into the culture chamber 105. A flow outlet 192 is correspondingly arranged in the wall section 120 to output culture medium 170 from the culture chamber 105. The flow inlet 190 and/or the flow outlet 192 is then preferably connected to a pump (not illustrated) to affect the flow of fresh culture medium 170 into the culture chamber 105 and removal of culture medium 170 therefrom. This embodiment is in particular advantageous if there is a desire to add any specific substance to the culture medium 170 during cell culturing and/or changing constituents or ingredients of the culture medium 170. The flow inlet 190 can then simply be connected to the new culture medium source or the new substance can be added to the culture medium source connected to the flow inlet 190.

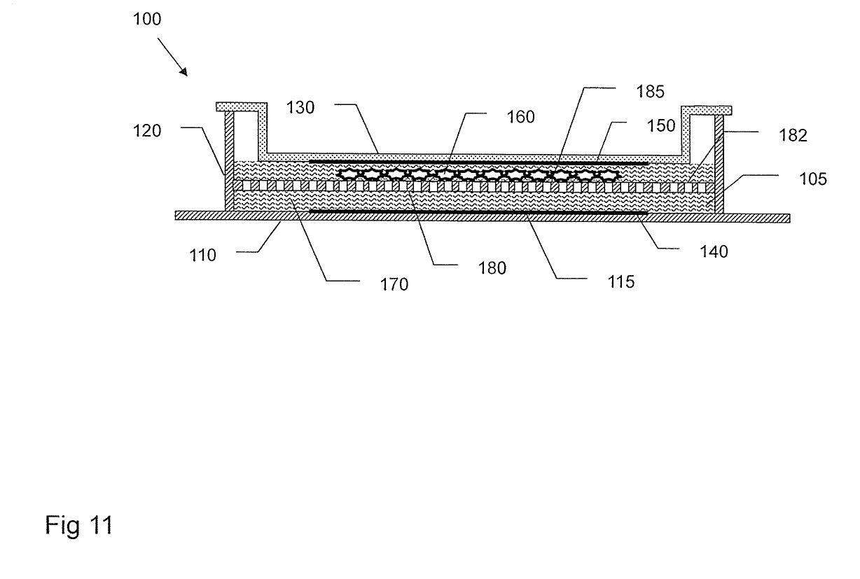

FIG. 11 is a cross-sectional view of another embodiment of a cell culture device 100. The cell culture device 100 comprises at least one intermediate culture plate 180, one such intermediate culture plate 180 is shown in FIG. 11. The intermediate culture plate 180 is attached to the wall section 120, such as resting on a shoulder of wall section 120 so that the intermediate culture plate 180 is removable from the wall section 120. Alternatively, the intermediate culture plate 180 could be mechanically attached to the wall section 120 so that it is not removable therefrom. At this position the intermediate culture plate 180 is present inside the culture chamber 105 and arranged between the culture bottom 110 and the lid 130. At least a portion of the top surface of the intermediate culture plate 180, i.e. the surface facing the lid 130, constitutes the cell culture area 185 of the cell culture device 100.

As is seen in FIG. 5, the current density within the culture chamber 105 is homogenous and does not change when moving from one of the electrodes 140, 150 to other electrode 140, 150. Hence, cells 160 cultured on the cell culture area 185 of the intermediate culture plate 180 in FIG. 11 will be exposed to substantially the same homogenous current density as cells cultured on the cell culture area 115 of the chamber bottom 110 in FIG. 4.

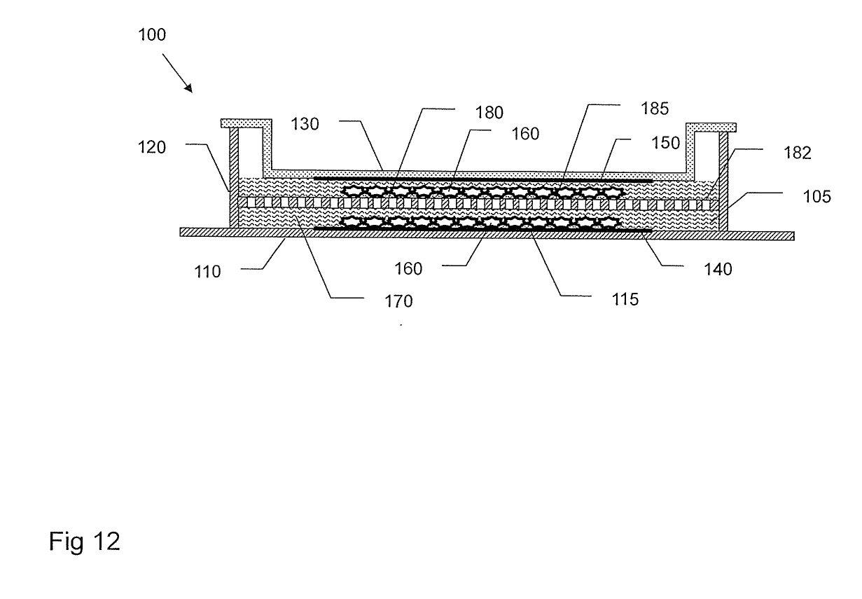

It is also possible to have multiple separate cell culture areas 115, 185 in the culture chamber 105 as is shown in FIG. 12. Thus, a first cell culture area 185 is present on the top surface of the intermediate culture plate 180 and a second cell culture area 115 is present on the top surface of the chamber bottom 110. This embodiment thereby basically doubles the amount of cells 160 that can be cultured in the cell culture device 100 as compared to the embodiments illustrated in FIG. 4 or 11. This approach can be extended further if using more than one intermediate culture plate 180 attached to the wall section 120 and arranged in between the culture bottom 110 and the lid 130.

The intermediate culture plate 180 could be made of an electrically insulating material, such as glass or a plastic material and in particular a transparent glass or plastic material. However, the intermediate culture plate 180 is designed to be electrically transparent in at least the portion of the intermediate culture plate 180 that carries the first cell culture area 185 or is aligned with the second cell culture area 115, mentioned above. Electrical transparency implies that current can pass through at least this portion of the intermediate culture plate 180. Hence, the intermediate culture plate 180 will then not negatively affect the electrical field and the current density profile even if it is made of an electrically insulating material. This can be achieved if the intermediate culture plate 180 is perforated and comprises multiple through holes 182 to enable current propagation through the intermediate culture plate 180 when stimulation pulses are applied to the first and second electrodes 140, 150. The through holes 182 need only to be sufficiently large to enable such current propagation. However, it is generally preferred if the through holes 182 are sufficiently large to enable the culture medium 170 to move through the intermediate culture plate 180 in order to have fluid connection between the upper compartment of the culture chamber 105 between the lid 130 and the intermediate culture plate 180 and the lower compartment of the culture chamber 105 between the intermediate culture plate 180 and the chamber bottom 110.

The through holes 182 are present in at least the portion of the intermediate culture plate 180 comprising or aligned with the cell culture area(s) 115, 185. The complete intermediate culture plate 180 could, though, be perforated. The through holes 182 preferably have a diameter that is sufficiently small to prevent the cells 160 from passing through the through holes 182.

Other solutions to achieve electrical transparency of the intermediate culture plate 180 include using a sintered material or a material comprising electrodes enabling electrical connection between the upper compartment of the culture chamber 105 and the lower compartment of the culture chamber 105.

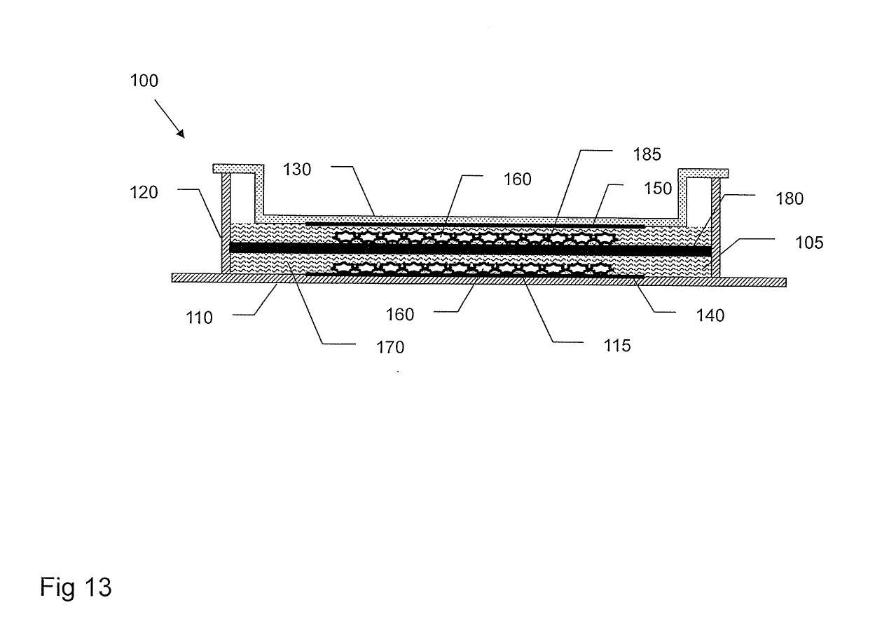

FIG. 13 illustrates another embodiment of a cell culture device 100 comprising an intermediate culture plate 180 made of or comprising an electrically conductive material, such as any of the previously mentioned metallic materials that can be used for the first and second electrode 140, 150. If the intermediate culture plate 180 is made of the electrically conductive material or comprises electrically conductive material that passes through the thickness of the intermediate culture plate 180 then no through holes are needed in the intermediate culture plate 180 in order for the current to propagate through the intermediate culture plate 180. In the latter case, at least the portion of the intermediate culture plate 180 aligned with the cell culture area(s) 115, 185 are preferably made of the electrically conductive material.

It is though possible to use at least one through hole in the intermediate culture plate 180 of the embodiment illustrated in FIG. 13 in order to achieve a fluid connection between the upper and lower compartments of the culture chamber 105.

The intermediate culture plate 180 could also be connected to the stimulation pulse generator 400 illustrated in FIG. 4 to thereby apply stimulation pulses over the first electrode 140, the second electrode 150 and the intermediate culture plate 180 to form electric fields between the first electrode 140 and the intermediate culture plate 180 and between the second electrode 150 and the intermediate culture plate 180. These electric fields then have a respective current density level within the target current density interval in the culture areas 115, 185 of the chamber bottom 110 and the intermediate culture plate 180, respectively.

In a further variant the intermediate culture plate 180 is made of an electrically insulating material with a first plate or sheet of electrically conductive material attached to its top surface facing the lid 130 and a second plate or sheet of electrically conductive material attached to its bottom surface facing the chamber bottom 110. In such an embodiment, it is possible to generate two electric fields, one in each compartment of the culture chamber 105.

The two plates or sheets are then preferably interconnected or each is connected to the stimulation pulse generator 400.

The embodiments of the cell culture device 100 discussed above and disclosed in FIGS. 4, 11-14 can be combined. For instance, the flow inlet 190 and flow outlet 192 as shown in FIG. 14 can be arranged in the cell culture device 100 according to any of the FIGS. 4, 11-13. The particular design of the first and second electrodes 140, 150 discussed in the foregoing in connection with FIGS. 6-8 can be applied to any of the embodiments of the cell culture device 100 shown in FIGS. 4, 11-14.

The stimulation pulse generator 400 indicated in FIG. 4 forms, in an embodiment, part of the cell culture device 100.

The stimulation pulse generator 400 is preferably configured to apply stimulation pulses over the first and second electrodes 140, 150 to form the electric field with a current density level within a target current interval of from about 150 A/m² to about 400 A/m². In addition, the particular design of the cell culture device 100 enables formation of an electric field with a homogenous and even current density distribution and profile in the cell culture area 115, 185 and where the current density is within the above specified preferred interval.

In an embodiment, the stimulation pulse generator 400 is configured to apply stimulation pulses with a duration in an interval of from about 0.1 to about 10 ms. Generally, a longer pulse duration implies that a lower pulse amplitude (V) can be used for the stimulation pulses and still induce the desired response in the cells 160, i.e. contraction or differentiation towards contracting ability. The stimulation pulses are preferably applied at an interval of about 200 to about 2000 ms to the first electrode 140 and the second electrode 150.

The stimulation pulses could be monophasic (negative or positive) pulses but it is generally preferred to use biphasic and in particular triphasic or quadriphasic waveforms (negative and positive) for the stimulation pulses. Even higher number of phase changes is possible. Triphasic and quadriphasic waveforms are more efficient in inducing contraction in cells 160 present in the cell culture device 100 as compared to mono- or biphasic waveforms since they allow for usage of lower voltage amplitudes and lower supplied energy.

The cell culture device 100 of the embodiments preferably enables control of the temperature in the culture chamber 105. This can be achieved by the introduction of controllable heating elements (not illustrated) present in the culture chamber 105 to thereby heat the culture medium 170 to a desired target temperature. A temperature sensor (not illustrated) is then advantageously arranged inside the culture chamber 105, such as attached to the wall section 120, the chamber bottom 110 or lid 130 to give a feedback signal representing a current temperature of the culture medium. The level of heating applied by the controllable heating elements is then controlled based on this feedback signal.

Alternatively, or in addition, the cell culture device 100 could be placed inside a room or facility having a controlled temperature to thereby get the desired culturing temperature inside the culture chamber 105.

Alternatively, or in addition, if the cell culture device 100 comprises a flow inlet 190 and a flow outlet 192 as illustrated in FIG. 14 the culture medium 170 input to the culture chamber 105 can be pre-tempered to the desired target temperature prior to flowing into the culture chamber 105.

The surface(s) of the cell culture area(s) 115, 185 in the cell culture device 100 can be untreated surfaces of the first electrode 140 or the chamber bottom 110, and the intermediate culture plate 180. Alternatively, these surfaces can be treated according to techniques well known within the technical field in order to promote cell anchoring and culturing. For instance, the surfaces can be chemically treated or be coated with various substances, such as fibronectin commonly used for cell culturing.

The cell culture device 100 of the embodiments not only provides a very homogenous current density at the cell culture area(s) 115, 185 of the culture chamber 105. The particular design of the cell culture device 100 also enables accurate determination and verification of the current density in the culture chamber 105. Hence, the present embodiments enable, in an efficient way, control of the stimulation environment in the culture chamber 105 to achieve a desired target current density.

The cell culture device 100 can be designed to have dimensions suitable for experimental set-ups but is also easy to scale up for production of large quantities of cells differentiated to have contracting ability.

Although modifications and changes may be suggested by those skilled in the art, it is the intention of the inventors to embody within the patent warranted hereon all changes and modifications as reasonably and properly come within the scope of their contribution to the art.

We claim as our invention:

1. A cell culture device comprising:
a chamber bottom;
a wall section attached to said chamber bottom and delimiting a culture chamber having a cell culture area onto which cells are to be cultured;
a lid attachable to said wall section to enclose said culture chamber;
a first electrode provided in or attached to said chamber bottom and extending over at least said cell culture area; and
a second electrode provided in or attached to said lid to be aligned with said first electrode when said lid is attached to said wall section, said second electrode extending over an area in said lid substantially corresponding to at least said cell culture area, wherein said first electrode and said second electrode are connectable to a stimulation pulse generator configured to apply stimulation pulses between said first electrode and said second electrode to form an electric field between said first electrode and said second electrode with a current density level within a target current density interval in said cell culture area.

2. The cell culture device according to claim 1, wherein said first electrode is a plate electrode of an electrically conductive material provided in or attached to said chamber bottom.

3. The cell culture device according to claim 2, wherein said first electrode comprises at least one through hole.

4. The cell culture device according to claim 3, wherein said at least one through hole of said first electrode has a longest dimension that is smaller than a distance between said first electrode and said second electrode when said lid is attached to said wall section.

5. The cell culture device according to claim 1, wherein said second electrode is a plate electrode of an electrically conductive material provided in or attached to said lid.

6. The cell culture device according to claim 5, wherein said second electrode comprises at least one through hole.

7. The cell culture device according to claim 6, wherein said at least one through hole of said second electrode has a longest dimension that is smaller than a distance between said first electrode and said second electrode when said lid is attached to said wall section.

8. The cell culture device according to claim 1, wherein said first electrode is a plate electrode of an electrically conductive material provided in or attached to said chamber bottom and comprises at least one through hole and said second electrode is a plate electrode of an electrically conductive material provided in or attached to said lid and comprises at least one through hole to be aligned with said at least one through hole of said first electrode when said lid is attached to said wall section.

9. The cell culture device according to claim 1, wherein said second electrode is in the form of a net of an electrically conductive material provided in or attached to said lid.

10. The cell culture device according to claim 1, wherein said second electrode is in the form a matrix of electrically inter-connected dots of an electrically conductive material provided in or attached to said lid.

11. The cell culture device according to claim 1, wherein said first electrode extends substantially over a whole bottom surface of said culture chamber.

12. The cell culture device according to claim 1, wherein said second electrode extends over said area in said lid corresponding to an area that said first electrode extends over in said chamber bottom.

13. The cell culture device according to claim 1, wherein said cell culture device comprises said stimulation pulse generator connected to said first electrode and said second electrode.

14. The cell culture device according to claim 13, wherein said stimulation pulse generator is configured to apply stimulation pulses between said first electrode and said second electrode to form said electric field with said current density level within said target current interval of 150 to 400 A/m² in said cell culture area.

15. The cell culture device according to claim 13, wherein said stimulation pulse generator is configured to apply one of triphasic and quadriphasic stimulation pulses between said first electrode and said second electrode to form said electric field with a homogenous current density level in said cell culture area.

16. The cell culture device according to claim 13, wherein said stimulation pulse generator is configured to apply stimulation pulses with a duration in an interval of from 0.1 to 10 ms once every 200 to 2000 ms between said first electrode and said second electrode.

17. The cell culture device according to claim 1, further comprising:
a flow inlet arranged in said wall section to input culture medium into said culture chamber; and
a flow outlet arranged in said wall section to output culture medium from said culture chamber.

18. The cell culture device according to claim 1, wherein said chamber bottom, said wall section and said lid are made of an electrically insulating material.

19. The cell culture device according to claim 18, wherein said electrically insulating material is selected from glass or electrically insulating plastic materials.

20. The cell culture device according to claim 18, wherein said electrically insulating material is transparent.

21. The cell culture device according to claim 1, further comprising an intermediate culture plate attached to said wall section and arranged in between said culture bottom and said lid, at least a portion of a surface of said intermediate culture plate constitutes said cell culture area.

22. The cell culture device according to claim 21, wherein said at least a portion of said surface of said intermediate culture plate constitutes a first cell culture area and said chamber bottom has a surface with a second cell culture area that is substantially aligned with said first cell culture area.

23. The cell culture device according to claim 21, wherein said intermediate culture plate is made of an electrically insulating material and comprises multiple through holes in a portion of said intermediate culture plate that constitutes said cell culture area.

24. The cell culture device according to claim 21, wherein said intermediate culture plate is made of an electronically conductive material.

25. The cell culture device according to claim 24, wherein said first electrode, said second electrode and said intermediate culture plate are connectable to said stimulation pulse generator configured to apply stimulation pulses between said first electrode, said second electrode and said intermediate culture plate to form a first electric field between said first electrode and said intermediate culture plate and a second electric field between said intermediate culture plate and said second electrode, wherein said first electric field and said second electric field have a respective current density level within said target current density interval in said culture area of said chamber bottom and said culture area of said intermediate culture plate.
